(19) United States
(12) Patent Application Publication
(10) Pub. No.: US 2020/0078575 A1
(43) Pub. Date: Mar. 12, 2020

(54) METHODS AND APPARATUS FOR SUPPORTING MICRONEEDLES

(71) Applicant: Microdermics Inc., Vancouver (CA)

(72) Inventors: Iman Mansoor, West Vancouver (CA); Sahan Anupama Ranamukhaarachchi, Vancouver (CA); Mehrsa Raeiszadeh, North Vancouver (CA); Boris Stoeber, Vancouver (CA)

(21) Appl. No.: 16/575,301

(22) Filed: Sep. 18, 2019

Related U.S. Application Data
(63) Continuation of application No. PCT/CA2018/050300, filed on Mar. 13, 2018.
(60) Provisional application No. 62/474,961, filed on Mar. 22, 2017.

(51) Int. Cl. A61M 37/00

(52) U.S. Cl. CPC A61M 37/0015; A61M 2037/0007; A61N 1/327; A61M 2037/003; A61M 2037/0061; A61M 2037/0023

(57) ABSTRACT

Methods and apparatus for supporting microneedles are provided. The apparatus includes a plurality of pedestals extending away from a base and transversely spaced-apart from each other by inter-pedestal volumes. Each of the pedestals has a transversely extending contact surface. For each of the pedestals, one or more microneedles extend from the contact surface of the pedestal.

### CROSS-REFERENCE TO RELATED APPLICATIONS

This application is a continuation of Patent Cooperation Treaty (PCT) application No. PCT/CA2018/050300 having an international filing date of 13 Mar. 2018. PCT application No. PCT/CA2018/050300 in turn claims priority, and the benefit under 35 U.S.C. § 119, from US application No. 62/474961 filed 22 Mar. 2017. All of the applications referenced in this paragraph are hereby incorporated herein by reference for all purposes.

### TECHNICAL FIELD

This invention relates to methods and apparatus for supporting microneedles. In particular, this invention relates to methods and apparatus for supporting microneedles in a manner which improves the efficacy of the microneedles for penetrating at least outer layers of tissue and/or for delivery of treatment (e.g. depositing treatment fluids, applying electrical signal and/or the like) to such tissue.

### BACKGROUND

Microneedles and methods for fabricating microneedles are disclosed, for example, in Kim et al., A tapered hollow metallic microneedle array using backside exposure of SU-8, (2001), J. Micromech. Microeng., Vol. 14, no. 4, pp. 597-603 and in PCT application No. PCT/CA2014/050552 filed 12 Jun. 2014. Both of the references of the preceding sentence are hereby incorporated herein by reference.

US Patent Publication No. 2009/0012494 (Yeshurun et al.) describe a prior art support for a plurality of microneedles.

There is a general desire for effective methods and apparatus for supporting microneedles. There is a further desire for effective methods and apparatus for supporting microneedles in a manner which improves the efficacy of the microneedles for penetrating at least outer layers of tissue and/or for delivery of treatment (e.g. depositing treatment fluids, applying electrical signal and/or the like) to such tissue.

### SUMMARY

The following embodiments and aspects thereof are described and illustrated in conjunction with methods and apparatus which are meant to be exemplary and illustrative, not limiting in scope.

One aspect of the invention provides an apparatus for supporting microneedles. The apparatus includes a plurality of pedestals extending away from a base. The pedestals are transversely spaced-apart from each other by inter-pedestal volumes. Each of the pedestals has a transversely extending contact surface. For each of the pedestals, one or more microneedles extend from the contact surface. Upon application of pressure by the apparatus to tissue, the contact surfaces of the pedestals may contact the tissue to apply forces to the tissue.

In some embodiments, the plurality of pedestals extend axially from the base in an axial direction. The axial direction may be generally orthogonal to the transversely extending contact surface.

In some embodiments, the one or more microneedles extend axially from the contact surface in an axial direction. In some embodiments, the one or more microneedles extend in directions with one or more transverse components (in addition to the axial component) as they extend axially away from the contact surface.

In some embodiments, the one or more microneedles extending from the contact surface of each pedestal includes a plurality of microneedles extending from the contact surface of each pedestal. The plurality of microneedles may be transversely spaced-apart from each other by inter-needle volumes.

In some embodiments, the microneedles provide a fluid path. In some embodiments, the microneedles do not provide a fluid path. In some embodiments, the microneedles are made from metal, silicon, glass and polymer.

In some embodiments, the base is in fluid communication with a fluid reservoir and a fluidic path defined by each pedestal. Fluid may be delivered to, or extracted from, the one or more microneedles extending from each pedestal.

In some embodiments, each microneedle defines an aperture. The aperture may be in fluid communication with the fluidic path defined by its corresponding pedestal.

In some embodiments, the fluidic paths of each of the plurality of pedestals are in fluid communication with one another. In some embodiments, the fluidic paths of the plurality of pedestals are independent from (not in fluid communication) with one another.

In some embodiments, the one or more microneedles that extend from the contact surface of at least one pedestal includes a plurality of microneedles, and the fluidic paths of the at least one pedestal may be in fluid communication with one another.

In some embodiments, the one or more microneedles that extend from the contact surface of at least one pedestal includes a plurality of microneedles, and the fluidic paths of the at least one pedestal are independent (not in fluid communication) with one another.

In some embodiments, the base is releasably coupled to the fluid reservoir. The fluid reservoir may be a syringe or any one of a prefilled cartridge, a deformable pouch or a rigid container sealed with a flexible wall such as a membrane. In some embodiments, the fluid reservoir is directly integrated with the apparatus.

In some embodiments, the base has an elevated region that carries the pedestals surrounded by a lower region.

In some embodiments, the base includes a customized fitting or a standard fitting on the side connecting to the fluid reservoir. Standard fitting may include a Luer Lock.

In some embodiments, the base is operatively connected to one or more sources of electric power for transmitting electric power to the one or more microneedles that extend from each pedestal.

In some embodiments, each pedestal includes one or more electrically conductive paths from the one or more sources of electric power to the one or more microneedles that extend from the contact surface of the pedestal.

In some embodiments, the electrically conductive paths of each of the plurality of pedestals are electrically insulated from one another (e.g., the electrically conductive paths of the plurality of pedestals are connected to different electric power sources). In some embodiments, the electrically conductive paths of each of the plurality of pedestals are electrically connected to one another.

In some embodiments, the one or more microneedles that extend from the contact surface of at least one pedestal includes a plurality of microneedles and the electrically conductive paths of the at least one pedestal are electrically insulated from one another.

In some embodiments, the one or more microneedles that extend from the contact surface of at least one pedestal includes a plurality of microneedles and the electrically conductive paths of the at least one pedestal are electrically connected to one another.

In some embodiments, the base is releasably coupled to the one or more sources of electric power.

In some embodiments, the at least one of the plurality of pedestals is positioned near an edge of the base (e.g. within 20% of a maximum cross-sectional dimension of the base in some embodiments or within 10% of a maximum cross-sectional dimension of the base in some embodiments).

In some embodiments, the one or more microneedles is positioned near an edge of the contact surface (e.g. within 5 times the axial extent of the one or more microneedles from the contact surface in some embodiments or within 2 times the axial extent of the one or more microneedles from the contact surface in some embodiments).

In some embodiments, the pedestal tapers, from transversely larger to transversely smaller, as it extends away from the base.

In some embodiments, the pedestals positioned at the outermost transverse position on the base may each have a contact surface that is transversely larger than the contact surface of the other pedestals positioned between the outermost positioned pedestals. The axial height of the pedestals and the transverse width of the contact surfaces may be the same as or different from that of adjacent pedestals.

In some embodiments, the pedestal is cylindrically shaped in cross-section. In some embodiments, the pedestal is elliptical shaped in cross-section. In some embodiments, the pedestal is polygonally shaped in cross-section.

In some embodiments, the plurality of pedestals 14 extending from base 12 comprise different heights such that their respective contact surfaces 20 are not located in one plane (e.g., some or all contact surfaces 20 having different distances from base 12 relative to one another). Specifically, where there are a plurality of pedestals, the contact surfaces of the pedestals may be located at different axial distances from the base.

In some embodiments, the contact surfaces are planar. In some embodiments, the contact surfaces need not be planar and the contact surfaces may have other surface profiles.

In some embodiments, a microneedle is positioned at a center of the pedestal. In some embodiments, three microneedles are positioned near the corners of a pedestal that is triangular shaped in cross-section.

In some embodiments, the pedestal is fabricated using conventional machining such as milling, electroplating, and injection molding. In some embodiments, the pedestal is fabricated using microfabrication methods such as photolithography and etching. In some embodiments, the pedestal is fabricated using rapid prototyping methods such as extrusion and stereolithography.

Another aspect of the invention provides one or more imprinting structures for use with a microneedle support apparatus. Each imprinting structure may have one or more first surfaces for contacting a tissue of a skin and one or more open regions therethrough. Each open region may be aligned to receive one or more pedestals. Upon application of pressure by the imprinting structure to tissue, the one or more first surfaces may contact the tissue to apply forces to the tissue which forces may cause the tissue to deform into the one or more open regions. The imprinting structure may facilitate deeper penetration of microneedles into the tissue, and may help to keep the liquid deposits formed spatially separated (by preventing the injected fluid from entering the compressed regions).

In some embodiments, the imprinting structure is placed on a surface of the tissue before microneedle application. In some embodiments, the imprinting structure is placed simultaneously or after microneedle application.

In some embodiments, the imprinting structure includes one continuous surface that is applied against the tissue. In some embodiments, the imprinting structure includes a plurality of surfaces that are applied against the tissue.

In some embodiments, the one or more imprinting structures are releasably coupleable to a plurality of pedestals that extend from a base of a support apparatus.

Another aspect of the invention provides a method for using microneedles to penetrate into at least an outer layer of a tissue of a patient. The method may include pressing a plurality of pedestals against a surface of the tissue. The pedestals may be transversely spaced-apart by inter-pedestal volumes. Each pedestal may be supporting one or more microneedles on a transversely-extending contact surface. The pressing of the pedestals against the surface of the tissue may cause an elastic deformation of the tissue into the inter-pedestal volumes.

In some embodiments, the method further includes injecting fluid into the tissue through apertures defined in each of the one or more microneedles. The injection of fluid into the tissue by using this method results in the formation of a plurality of spatially separated fluid deposits in the tissue. In some embodiments, fluid is injected into skin tissue. In such embodiments, the fluid deposits are formed in the skin's dermis or epidermis layers.

In some embodiments, the fluid deposits formed in the tissue during one injection procedure are different in size and hold different fluid volumes. In some embodiments, individual fluid deposits are formed. In some embodiments, a connected deposit region is formed when a larger amount of fluid is delivered.

In some embodiments, the fluid is a therapeutic compound. In some embodiments, the fluid contains particles.

In some embodiments, the method includes extracting fluid from the tissue through the fluidic path. In some embodiments, the method includes delivering electrical power to the tissue through each of the one or more microneedles.

In some embodiments, the method includes providing a coating material to the one or more microneedles. The coated material may be transferred into tissue by dissolving or opening up pores.

Another aspect of the invention provides a method of using one or more imprinting structures with a microneedle support apparatus to penetrate microneedles into at least an outer layer of a tissue of a patient. The method includes positioning one or more imprinting structures on a surface of the tissue. Each imprinting structure has a first surface and at least one open region therethrough. The method further includes inserting one or more pedestals through one open region of the one or more imprinting structures and applying a force to the one or more first surfaces in a direction toward the surface of the tissue. The application of the force to the one or more first surfaces may cause an elastic deformation of the tissue into the open regions.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIGS. 1A-C (collectively, FIG. 1) are perspective views illustrating different configurations of a microneedle support apparatus according to example embodiments.

FIG. 2 is a perspective view illustrating the FIG. 1 microneedle support apparatus connected to a syringe.

FIG. 3A is a schematic diagram showing the FIG. 1 microneedle support apparatus placed on a surface of a tissue of a patient before microneedle application. FIG. 3B is a schematic diagram showing the FIG. 1 microneedle support apparatus during microneedle application.

FIGS. 4A and 4B (collectively, FIG. 4) are perspective views illustrating different configurations of an imprinting structure for use in conjunction with the FIG. 1 microneedle support apparatus according to example embodiments.

FIG. 5A is a schematic diagram showing the FIG. 4 imprinting structure positioned on a surface of a tissue of a patient before microneedle application. FIG. 5B is a schematic diagram showing the FIG. 4 imprinting structure used in conjunction with the FIG. 1 microneedle support apparatus during microneedle application.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

This disclosure and the accompanying claims relate to the delivery of spatially separated fluid deposits in tissue which involves using a support apparatus for microneedles that provides spatially separated paths. The support apparatus comprises a plurality of transversely-spaced pedestals. The plurality of transversely-spaced pedestals is separated from each other by inter-pedestal volumes (i.e., void spaces). Each pedestal comprises a transversely extending contact surface. For each of the pedestals, one or more microneedles extend from the contact surface of the pedestal. Application of microneedles supported on transversely-spaced pedestals onto a tissue surface causes elastic deformation of the tissue into the inter-pedestal volumes. This allows for the delivery of spatially separated wheals (or fluid deposits) or spatially separated current paths in the tissue. Some of the benefits of this invention include:
Improve penetration of the microneedle as the skin stretches into inter-pedestal volumes. The stratum corneum can more easily reach its tensile stress or strain limit if it is stretched around microneedles and slender pedestals.
Faster absorption of liquid in skin. Fluid will be absorbed by the surrounding tissue from the surface surrounding the fluid deposit. The total area of the surrounding surface is larger for a number of smaller deposits compared to a single deposit of the same volume.
Less pain to a patient during injection since liquid is distributed in small deposits (less skin nerve compression).
Faster injection through the use of multiple microneedles as compared to the use of one microneedle. The resistance of skin to the injection of a fluid volume (backpressure) will increase with the amount of fluid injected; thus, smaller deposits will be associated with a smaller backpressure. The same pressure will be present in all deposits if the fluid paths to the different needles are connected because injection will then occur in parallel.

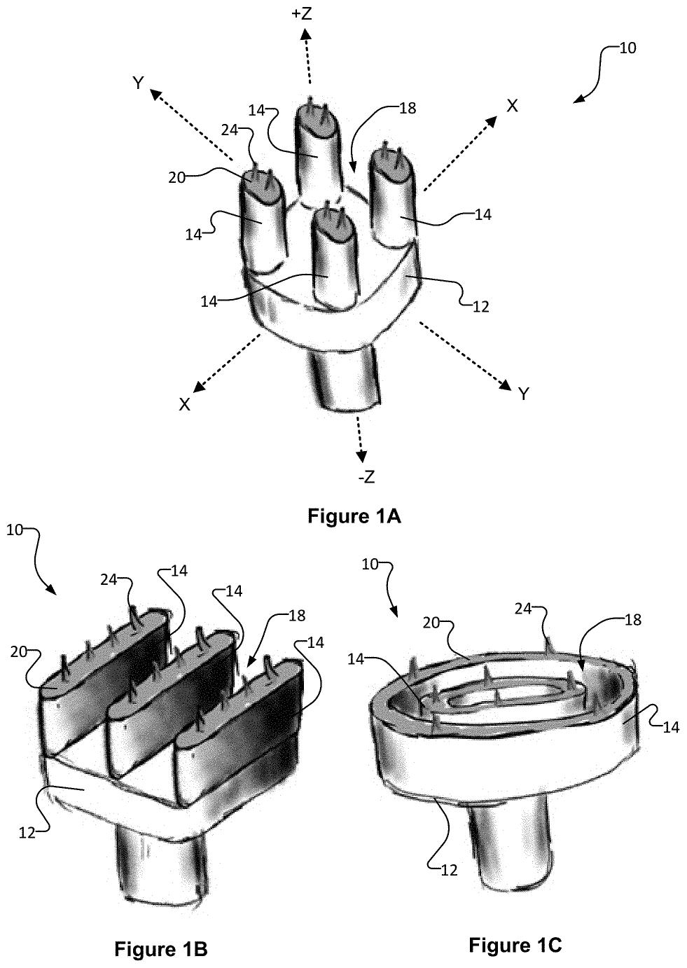

FIG. 1 illustrates different embodiments of the support apparatus for microneedles 10. Support apparatus 10 may comprise a base 12 and a plurality of pedestals 14. Base 12 may comprise an elevated region that carries the pedestals, surrounded by a lower region. The plurality of pedestals 14 extend away from base 12. In some embodiments, pedestals 14 may be configured to extend axially from base 12, i.e., in an axial direction that is generally orthogonal to the transversely extending contact surface. In some embodiments, pedestals 14 may be configured to extend at an angle with respect to base 12. The plurality of pedestals 14 extending from base 12 may comprise different axial heights relative to each other. The plurality of pedestals 14 may be transversely spaced-apart from one another by inter-pedestal volumes 18. Inter-pedestal volumes 18 are the void spaces between pedestals 14. Pedestals 14 may comprise a contact surface 20. Contact surface 20 may extend in a transverse direction. Contact surfaces 20 on the plurality of pedestals 14 extending from base 12 may comprise different transverse widths relative to each other. Each of contact surfaces 20 may comprise one or more microneedles 24. One or more microneedles 24 may extend from contact surface 20. In some embodiments, microneedles 24 extend axially from contact surface 20, i.e., in an axial direction that is generally orthogonal to the transversely extending contact surface 20; however, this is not mandatory. Microneedles 24 may extend at an angle with respect to contact surface 20. In embodiments in which a plurality of microneedles 24 extends from contact surface 20, the plurality of microneedles 24 may be transversely-spaced apart from one another (as shown in any of FIGS. 1A-C) such that void spaces (i.e., inter-needle volumes) may also be provided between microneedles 24 positioned on the same pedestal.

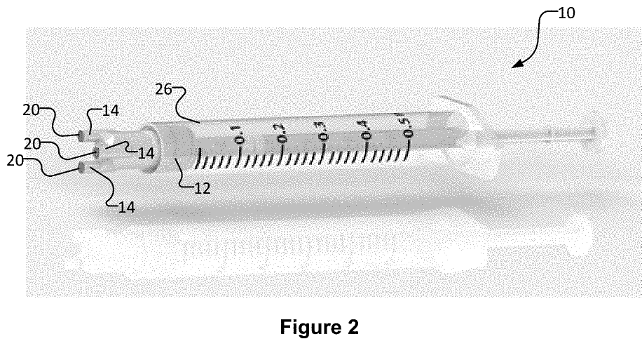

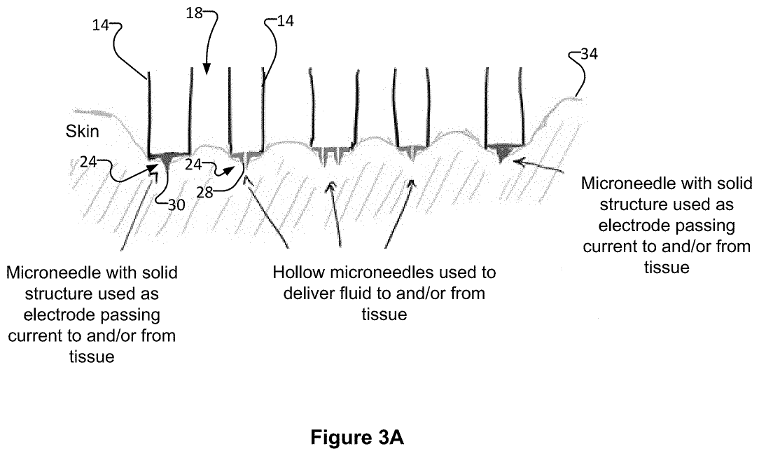

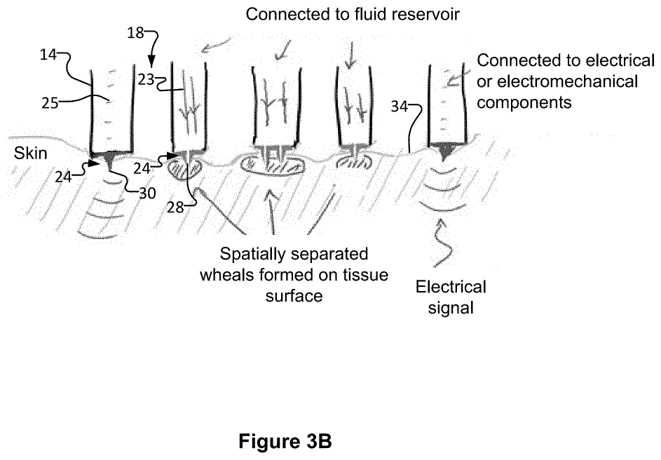

Referring to FIG. 3, pedestal 14 defines a fluidic path 23. Base 12 may be in fluid communication with a fluid reservoir 26 (shown in FIG. 2) and the fluidic path 23 of pedestal 24. Fluid may flow from fluid reservoir 26 through the fluidic path 23 of pedestal 24 to microneedle 24. Microneedle 24 may comprise an aperture 28. Aperture 28 may be in fluid communication with the fluidic path 23 of pedestal 14. Fluid may thus flow through a fluidic path 23 of pedestal 14 and exit pedestal 14 from aperture 28 for injection into a tissue. Fluid may also be extracted from the tissue.

In some embodiments, the multiple fluidic paths 23 of each of the plurality of pedestals 14 may be in fluid communication with one another. In some embodiments, the multiple fluidic paths 23 of pedestals 14 may be independent (not in fluid communication) with one another.

Pedestals 14 may be fabricated using any suitable conventional machining such as milling, electroplating and injection molding, microfabrication methods such as photolithography and etching, and rapid prototyping methods such as extrusion and stereolithography.

In some embodiments, base 12 may be releasably connected to fluid reservoir 26. In particular embodiments, fluid reservoir 26 is a syringe (as shown in FIG. 2). In such embodiments, base 12 may comprise customized fitting or a standard fitting, such as a Luer Lock, on the side for connecting to the syringe. Fluid reservoir 26 may, however, be any other suitable fluid carrying means. For example, fluid reservoir 26 may be a prefilled cartridge, a deformable pouch or a rigid container sealed with a flexible wall such as a membrane. In some embodiments, fluid reservoir 26 may be integrated with base 12.

Referring to FIG. 3A, microneedle 24 may comprise a solid tip 30. Solid tip 30 may not comprise an aperture for fluid flow-through. Solid tip 30 may comprise a hollow body. Either solid tip 30 or apertured tip 28 may be used as an electrode which passes current to or from tissue. Solid tip 30 may also be used to transfer coated materials into tissue, for example, by either dissolving or opening up pores. In such embodiments, base 12 may be operatively connected to one or more sources of electric power (not shown) for transmitting electric power to microneedle 24. Each pedestal 14 may comprise one or more electrically conductive path 25. Electrically conductive path 25 of each of the plurality of pedestals 14 may be electrically independent (e.g. insulated) from one another (e.g., electrically conductive paths 25 of the plurality of pedestals 14 may be connected to different electric potentials or may otherwise be independently electrically addressable). In some embodiments, electrically conductive path 25 of each of the plurality of pedestals 14 may be electrically connected to one another. This electric current may cause electroporation. Stimulation by electric current thus causes the enhancement of cell membrane permeability, which has at least the benefit of improving the absorption of drugs by the cells.

One embodiment of support apparatus 10 may include only apertured microneedles 24 supported by the plurality of pedestals 14. Another embodiment of support apparatus 10 may include only non-apertured (e.g. solid or hollow) microneedles 24 (i.e., microneedles that do not have apertures to allow fluid flow-through) supported by the plurality of pedestals 14. Another embodiment of a support apparatus 10 may include a combination of one or more apertured microneedles 24 and one or more solid-tip microneedles 24 supported by the plurality of pedestals 14.

Each microneedle 24 may either provide a fluid path (i.e., microneedle 24 having an aperture to allow fluid flow-through) or may not provide a fluid path (i.e., non-apertured microneedles). Microneedles 24 may be made from any suitable materials. Non-limiting exemplary materials include, metal, silicon, glass, ceramics, and polymer.

As illustrated in FIG. 1, pedestal 14 may comprise different configurations. Pedestal 14 may be cylindrical (e.g., FIG. 1A), elliptical (e.g., FIG. 1B), or polygonal shaped in cross section. Pedestals 14 may however comprise any other suitable shapes.

In some embodiments, pedestals 14 may be slender, i.e., having a relatively small width in transverse directions. Microneedles 24 may be positioned near an edge of contact surface 20. Microneedles 24 may be placed at any other suitable location on contact surface 20. For example, in one exemplary embodiment, a single microneedle 24 may be placed at the center of a slender pedestal. In another exemplary embodiment, three microneedles may be placed near the vertexes (or corners) of a pedestal 14 that comprises a triangular cross-sectional shape.

In particular embodiments, each pedestal 14 may taper from transversely larger to transversely smaller as it extends away from the base. This provides mechanical rigidity at the base and reduces the amount of potential pedestal bending.

In some embodiments, the plurality of pedestals 14 extending from base 12 comprise different heights such that their respective contact surfaces 20 are not located in one plane (e.g., some or all contact surfaces 20 having different distances from base 12 relative to one another). Specifically, where there are a plurality of pedestals 14, the contact surfaces 20 of the pedestals 14 may be located at different axial distances from the base 12.

The contact surfaces 20 need not be planar and the contact surfaces 20 may have other surface profiles.

FIG. 3 is a schematic diagram showing an exemplary method of using support structure 10. FIG. 3A shows support structure 10 placed on the surface of a tissue 34 (e.g., skin) before microneedle application. The plurality of pedestals 14 each supporting one or more microneedles 24 is pressed against the surface of the tissue 34 thereby causing microneedles 24 to be inserted into the tissue 34. When the microneedles 24 mounted on the transversely-spaced pedestals 14 are pressed against the tissue 34, the tissue 34 may nearly conform to the microneedles 24 and pedestals 14. In particular, the tissue 34 elastically deforms as a result of applying the pedestals 14 to the surface of the tissue 34. If the tissue 34 has an outer layer that can be ruptured in tension (at its tensile strength limit) then conformation of the tissue 34 around slender pedestals 14 and microneedles 24 will be associated with significant tensile stress or strain in the outer layer, in particular at the microneedle tips. As a result, this may lead to yielding of the outer layer at the needle tips. This effect can be observed when inserting microneedles 24 mounted on pedestals 14 against skin. In particular, the outermost layer of skin (e.g., stratum corneum) can rupture beyond its tensile strength limit.

FIG. 3B illustrates microneedle application after which support structure 10 comprising microneedles 24 mounted on the pedestals 14 is pressed against the surface of the tissue 34. In some embodiments, microneedle application involves injecting fluid into tissue 34. This involves injecting fluid from fluid reservoir 26 through fluidic paths 23 of pedestals 14 to one or more microneedles 24. As a result, spatially separated “wheals” (i.e., fluid deposits that may manifest themselves in the form of wheals which are observable from outside the tissue) may be formed on the tissue surface. For example, if fluid is injected into skin tissue, the fluid deposits may be formed in the skin's dermis or epidermis layers. The fluid deposits formed in the tissue during one injection may be different in size and hold different fluid volumes (as illustrated in FIG. 3B). In some embodiments, individual fluid deposits may be formed. In some embodiments, a connected deposit region may be formed when a larger amount of fluid is delivered. In particular embodiments, microneedle application involves transmitting an electrical current to one or more microneedles 24 through electrically conductive path 25 thereby, passing an electrical current to or from tissue 34.

Fluid may comprise any suitable materials. In some embodiments, fluid may comprise a therapeutic or cosmetic compound. In some embodiments, fluid may contain particles.

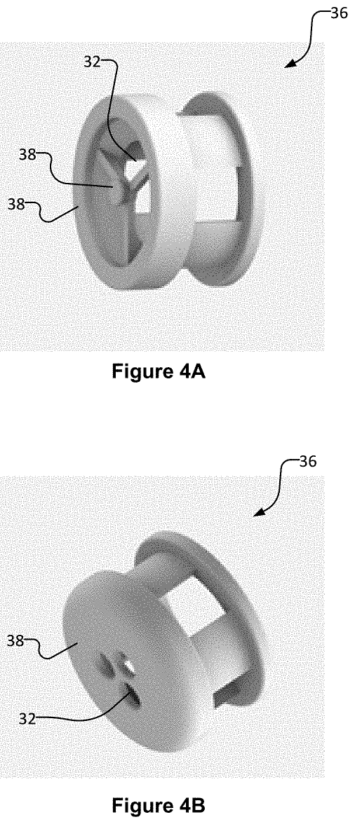

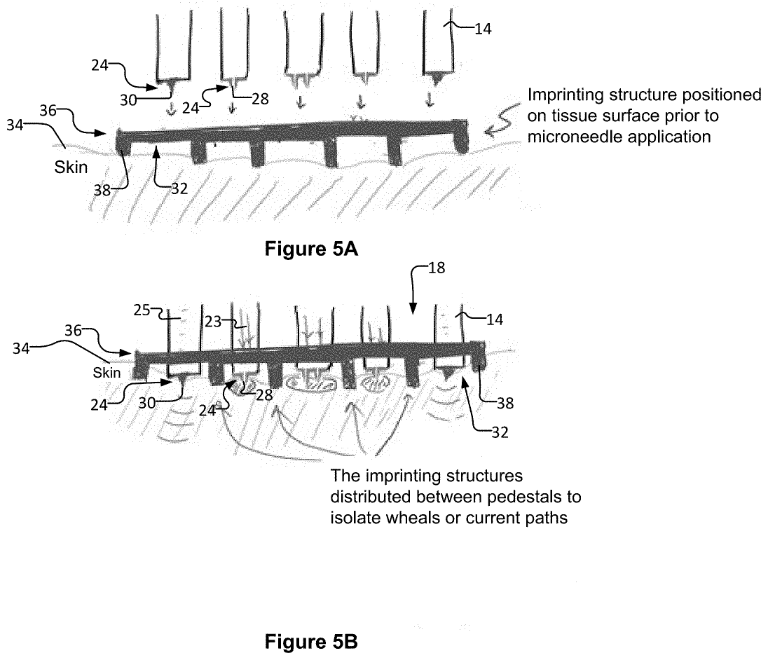

In particular embodiments, one or more imprinting structures 36 may be used in conjunction with support apparatus 10. FIG. 4 illustrates example embodiments of an imprinting structure 36. Imprinting structure 36 comprises an upper surface 38 and at least one open region 32 defined by upper surface 38. Each open region 32 is configured to align with one or more pedestals 14 (FIG. 5).

In some embodiments, upper surface 38 may be positioned on a surface of a tissue 34 prior to microneedle application (FIG. 5A). In some embodiments, upper surface 38 may be placed simultaneously or after microneedle application. A force may be asserted on upper surface 38 in the direction of the surface of the tissue 34. Without bound to any theory, the inventors believe that the use of imprinting structure 36 has at least the following advantages:
facilitates the insertion of the microneedles 24 on pedestals 14 into the tissue 34 as pressing the imprinting structure 36 against the tissue 34 will make the tissue 34 appear more firm;
facilitates deeper penetration of microneedles 24 in the tissue 34;
helps retain the liquid deposits that are formed spatially to be separated. This may occur when the imprinting structure 36 imparts pressure to the tissue 34 in such a way that it prevents the injected fluid from entering the compressed regions (i.e., the inter-pedestal volumes 18); and
provides an orientation of the tissue 34 in a perpendicular direction to the shaft of a microneedle 24 at the point of needle insertion.

Referring to FIG. 4B, imprinting structure 36 may include one continuous upper surface 38 that is applied against the surface of the tissue 34. Referring to FIG. 4A, imprinting structure 36 may comprise a plurality of upper surfaces 38.

FIG. 5 is a schematic diagram showing an exemplary method of using imprinting structure 36. FIG. 5A shows imprinting structure 36 being placed on the surface of a tissue 34 (e.g., skin) before microneedle application. FIG. 5B shows imprinting structure 36 being used in conjunction with support apparatus 10 during microneedle application. During microneedle application, a force may be asserted against the tissue 34 at upper surfaces 38. Each pedestal 14 may be received within an open region 32 of imprinting structure 36 such that upper surfaces 38 distribute between pedestals 14 to isolate wheals or current paths. The application of force of the upper surfaces 38 facilitates elastic deformation of the tissue 34 into the inter-pedestal volumes (i.e., the void spaces).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. An apparatus for supporting microneedles, the apparatus comprising:
a plurality of pedestals extending away from a base and transversely spaced-apart from each other by inter-pedestal volumes, each of the pedestals having a transversely extending contact surface;
for each of the pedestals, one or more microneedles which extend from the contact surface of the pedestal.

2. An apparatus according to claim 1 wherein, upon application of pressure by the apparatus to tissue, the contact surfaces contact the tissue to apply forces to the tissue which forces cause the tissue to deform into the inter-pedestal volumes.

3. An apparatus according to claim 1, wherein the plurality of pedestals extend axially from the base in an axial direction that is generally orthogonal to the transversely extending contact surface.

4. An apparatus according to claim 1, wherein the one or more microneedles extend axially from the contact surface in an axial direction that is generally orthogonal to the transversely extending contact surface.

5. An apparatus according to claim 1, wherein the one or more microneedles extending from the contact surface of each pedestal comprises a plurality of microneedles extending from the contact surface of each pedestal and the plurality of microneedles are transversely spaced-apart from each other by an inter-needle volume.

6. An apparatus according to claim 1, wherein the base is in fluid communication with a fluid reservoir and a fluidic path defined by each pedestal for delivering fluid to, or extracting fluid from, the one or more microneedles extending from each pedestal.

7. An apparatus according to claim 6, wherein each microneedle defines an aperture, the aperture being in fluid communication with the fluidic path defined by its corresponding pedestal.

8. An apparatus according to claim 6, wherein the fluidic paths of each of the plurality of pedestals are in fluid communication with one another.

9. An apparatus according to claim 6, wherein the fluidic paths of the plurality of pedestals are independent from (not in fluid communication) with one another.

10. An apparatus according to claim 6, wherein the one or more microneedles that extend from the contact surface of at least one pedestal comprises a plurality of microneedles and the fluidic paths of the at least one pedestal are in fluid communication with one another.

11. An apparatus according to claim 6, wherein the one or more microneedles that extend from the contact surface of at least one pedestal comprises a plurality of microneedles and the fluidic paths of the at least one pedestal are independent (not in fluid communication) with one another.

12. An apparatus according to claim 6, wherein the base is releasably coupled to the fluid reservoir.

13. An apparatus according to claim 1, wherein the base is operatively connected to one or more sources of electric power for transmitting electric power to the one or more microneedles extending from each pedestal.

14. An apparatus according to claim 13, wherein each pedestal comprises one or more electrically conductive paths from the one or more sources of electric power to the one or more microneedles that extend from the contact surface of the pedestal.

15. An apparatus according to claim 14, wherein the electrically conductive paths of each of the plurality of pedestals are electrically insulated from one another.

16. An apparatus according to claim 14, wherein the electrically conductive paths of each of the plurality of pedestals are electrically connected to one another.

17. An apparatus according to claim 14, wherein the one or more microneedles that extend from the contact surface of at least one pedestal comprises a plurality of microneedles and the electrically conductive paths of the at least one pedestal are electrically insulated from one another.

18. An apparatus according to claim 14, wherein the one or more microneedles that extend from the contact surface of at least one pedestal comprises a plurality of microneedles and the electrically conductive paths of the at least one pedestal are electrically connected to one another.

19. An apparatus according to claim 14, wherein the base is releasably coupled to the one or more sources of electric power.

20. An apparatus according to claim 1, wherein at least one of the plurality of pedestals is positioned near an edge of the base (e.g. within 20% of a maximum cross-sectional dimension of the base in some embodiments or within 10% of a maximum cross-sectional dimension of the base in some embodiments).

21.-66. (canceled)
